(19) United States
(12) Patent Application Publication
(10) Pub. No.: US 2010/0047306 A1
(43) Pub. Date: Feb. 25, 2010

(54) PHARMACEUTICAL COMPOSITIONS COMPRISING BISPHOSPONATES

(75) Inventors: Rolf Loeffler, Freiburg (DE); Holger Petersen, Eimeldingen (DE); Juergen Sigg, Loerrach (DE); Gesine Winzenburg, Inzlingen (DE)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 12/443,273

(22) PCT Filed: Oct. 3, 2007
(86) PCT No.: PCT/EP07/60508

§ 371 Date: Mar. 27, 2009

Related U.S. Application Data
(60) Provisional application No. 60/828,261, filed on Oct. 5, 2006.

(51) Int. Cl. A61K 31/663; A61K 9/14

(52) U.S. Cl. 424/422; 514/102; 424/486; 514/108

(57) ABSTRACT

The present invention relates to depot formulations comprising a poorly water soluble salt of a bisphosphonate forming together with one or more biocompatible polymers. The depot formulation may be in the form of microparticles or implants. The depot formulations are useful for the treatment and prevention of proliferative diseases including cancer.

### FIELD OF THE INVENTION

The present invention relates to depot formulations comprising a poorly water soluble salt of a bisphosphonate forming together with one or more biocompatible polymers. The depot formulation may be in the form of microparticles or implants. The depot formulations are useful for the treatment and prevention of proliferative diseases including cancer.

### BACKGROUND OF THE INVENTION

Bisphosphonates are widely used to inhibit osteoclast activity in a variety of both benign and malignant diseases in which bone resorption is increased. So far, only water soluble bisphosphonates, e.g., the sodium salt, have been used in pharmaceutical compositions. In case of forming solutions for infusion this is a reasonable approach. However, in case of a depot formulation the high water solubility of the bisphosphanate will lead to a high initial release causing severe local tissue irritations.

### SUMMARY OF THE INVENTION

It has now been surprisingly found that poorly water soluble bisphosphonates can be encapsulated very efficiently so that the drug release is very well under control.

The present invention relates to depot formulations comprising a poorly water soluble salt of a bisphosphonate forming together with biocompatible polymers.

The present invention relates to micropartices comprising a poorly water soluble salt of a bisphosphonate forming together with biocompatible polymers, preferably biodegradable polymers.

The present invention relates to implants comprising a poorly water soluble salt of a bisphosphonate forming together with biocompatible polymers.

The present invention relates to methods for the treatment and prevention of proliferative diseases, including cancer, comprising the depot formulations of the present invention.

### DETAILED DESCRIPTION OF THE DRAWINGS

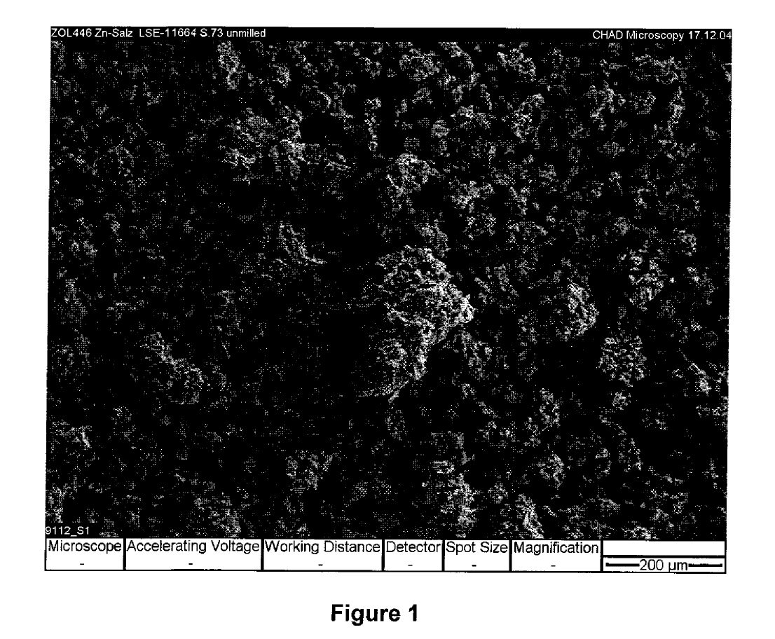

FIG. 1 illustrates scanning electron microscopy (SEM) images of unmilled zoledronic acid zinc salt.

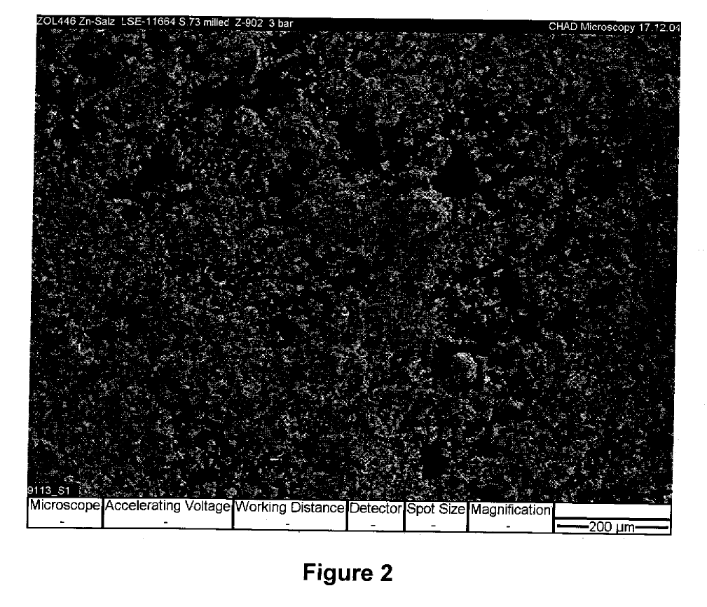

FIG. 2 illustrates SEM images of milled zoledronic acid zinc salt.

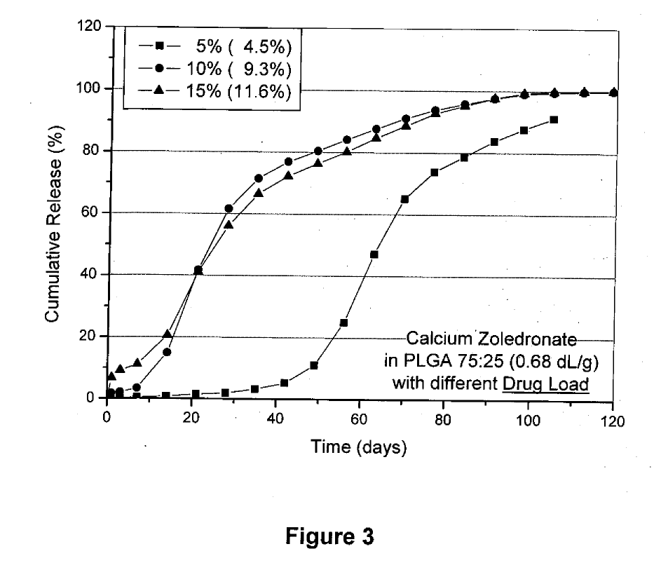

FIG. 3 illustrates the in vitro drug release of microparticles of zoledronic acid in the calcium salt form.

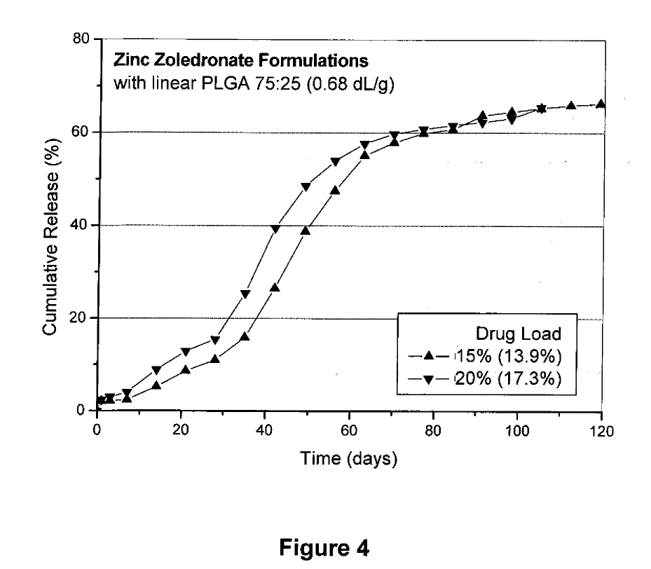

FIG. 4 illustrates the in vitro drug release of microparticles of zoledronic acid in the zinc salt form.

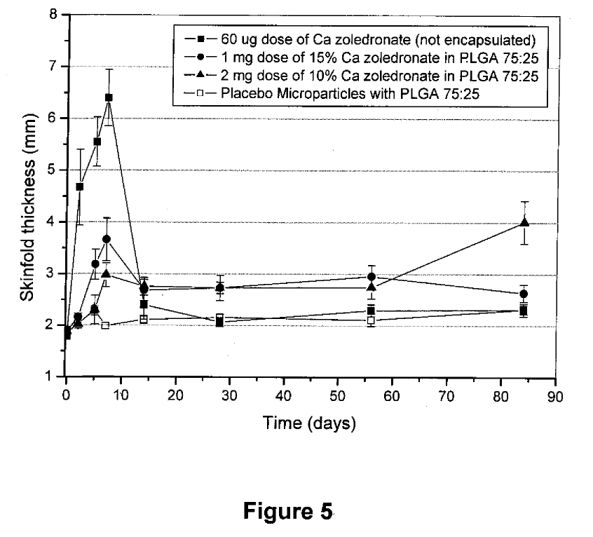

FIG. 5 illustrates the skin fold thickness after s.c. injection of calcium zoledronate not encapsulated and encapsulated in PLGA microparticles.

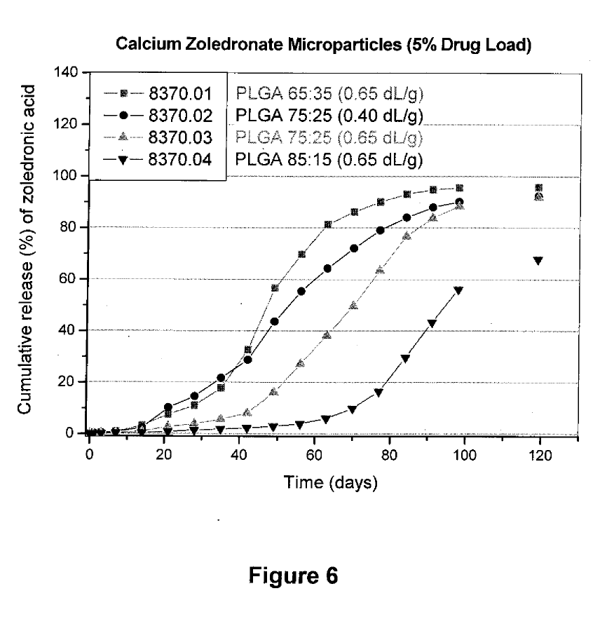

FIG. 6 illustrates the in vitro drug release of microparticles of zoledronic acid in the calcium salt form.

### DETAILED DESCRIPTION OF THE INVENTION

The present invention relates to depot formulations comprising a poorly water soluble salt of a bisphosphonate forming together with biocompatible polymers.

### I. Bisphosphonates

Examples of suitable bisphosphonates for use in the invention may include the following compounds or a pharmaceutically acceptable salt thereof: 3-amino-1-hydroxypropane-1,1-diphosphonic acid (pamidronic acid), e.g., pamidronate (APD); 3-(N,N-dimethylamino)-1-hydroxypropane-1,1-diphosphonic acid, e.g., dimethyl-APD; 4-amino-1-hydroxybutane-1,1-diphosphonic acid (alendronic acid), e.g., alendronate; 1-hydroxy-ethidene-bisphosphonic acid, e.g., etidronate; 1-hydroxy-3-(methylpentylamino)-propylidene-bisphosphonic acid, ibandronic acid, e.g., ibandronate; 6-amino-1-hydroxyhexane-1,1-diphosphonic acid, e.g., amino-hexyl-BP; 3-(N-methyl-N-n-pentylamino)-1-hydroxypropane-1,1-diphosphonic acid, e.g., methyl-pentyl-APD (=BM 21.0955); 1-hydroxy-2-(imidazol-1-yl)ethane-1,1-diphosphonic acid, e.g., zoledronic acid; 1-hydroxy-2-(3-pyridyl)ethane-1,1-diphosphonic acid (risedronic acid), e.g., risedronate, including N-methyl pyridinium salts thereof, e.g., N-methyl pyridinium iodides, such as NE-10244 or NE-10446; 1-(4-chlorophenylthio)methane-1,1-diphosphonic acid (tiludronic acid), e.g., tiludronate; 3-[N-(2-phenylthioethyl)-N-methylamino]-1-hydroxypropane-1,1-diphosphonic acid; 1-hydroxy-3-(pyrrolidin-1-yl)propane-1,1-diphosphonic acid, e.g., EB 1053 (Leo); 1-(N-phenylaminothiocarbonyl)methane-1,1-diphosphonic acid, e.g., FR 78844 (Fujisawa); 5-benzoyl-3,4-dihydro-2H-pyrazole-3,3-diphosphonic acid tetraethyl ester, e.g., U-81581 (Upjohn); 1-hydroxy-2-(imidazo[1,2-a]pyridin-3-yl)ethane-1,1-diphosphonic acid, e.g., YM 529; and 1,1-dichloromethane-1,1-diphosphonic acid (clodronic acid), e.g., clodronate.

A particularly preferred bisphosphonate for use in the invention comprises a bisphosphonate of compound of formula (I):

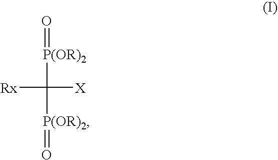

wherein
X is hydrogen, hydroxyl, amino, alkanoyl, or an amino group substituted by C₁-C₄ alkyl, or alkanoyl;
R is hydrogen or C₁-C₄ alkyl; and
Rx is a side chain which contains an optionally substituted amino group, or a nitrogen containing heterocycle (including aromatic nitrogen-containing heterocycles),
   or a pharmaceutically acceptable salt thereof or any hydrate thereof, embedded in a polymer matrix, wherein the pharmaceutically acceptable salt thereof, and wherein the pharmaceutically acceptable salt is selected from calcium, zinc and magnesium.

Examples of particularly preferred bisphophonates for use in the invention are:
2-(1-Methylimidazol-2-yl)-1-hydroxyethane-1,1-diphosphonic acid;
2-(1-Benzylimidazol-2-yl)-1-hydroxyethane-1,1-diphosphonic acid;
2-(1-Methylimidazol-4-yl)-1-hydroxyethane-1,1-diphosphonic acid;
1-Amino-2-(1-methylimidazol-4-yl)ethane-1,1-diphosphonic acid;
1-Amino-2-(1-benzylimidazol-4-yl)ethane-1,1-diphosphonic acid;
2-(1-Methylimidazol-2-yl)ethane-1,1-diphosphonic acid;
2-(1-Benzylimidazol-2-yl)ethane-1,1-diphosphonic acid;
2-(Imidazol-1-yl)-1-hydroxyethane-1,1-diphosphonic acid;
2-(Imidazol-1-yl)ethane-1,1-diphosphonic acid;
2-(4H-1,2,4-triazol-4-yl)-1-hydroxyethane-1,1-diphosphonic acid;
2-(Thiazol-2-yl)ethane-1,1-diphosphonic acid;
2-(Imidazol-2-yl)ethane-1,1-diphosphonic acid;
2-(2-Methylimidazol-4(5)-yl)ethane-1,1-diphosphonic acid;
2-(2-Phenylimidazol-4(5)-yl)ethane-1,1-diphosphonic acid;
2-(4,5-Dimethylimidazol-1-yl)-1-hydroxyethane-1,1-diphosphonic acid; and
2-(2-Methylimidazol-4(5)-yl)-1-hydroxyethane-1,1-diphosphonic acid,
   and pharmacologically acceptable salts thereof.

More preferred bisphosphonates for use in the invention are disodium-3-amino-1-hydroxy-propylidene-1,1-bisphosphonate pentahydrate (pamidronic acid); and 2-(imidazol-1yl)-1-hydroxyethane-1,1-diphosphonic acid (zoledronic acid), or pharmacologically acceptable salts thereof.

The most preferred bisphosphonate for use in the invention is 2-(imidazol-1yl)-1-hydroxyethane-1,1-diphosphonic acid (zoledronic acid), or a pharmacologically acceptable salt thereof.

The pharmacologically acceptable salt is selected from calcium, magnesium and zinc. These salts are low in water solubility.

The bisphosphonates of the invention may be prepared in accordance with conventional methods.

Preferably, the depot formulations of the invention contain as active ingredient only a compound of the invention, e.g., a compound of formula (I), preferably 2-(imidazol-1yl)-1-hydroxyethane-1,1-diphosphonic acid (zoledronic acid).

Preferably, the microparticles of the invention contain a compound of formula (I), in form of the calcium salt, even more preferably the calcium salt of 2-(imidazol-1yl)-1-hydroxyethane-1,1-diphosphonic acid (zoledronic acid).

The bisphosphonates of the invention may be present in an amount of from about 1% to about 60%, more usually about 2% to about 20%, preferably about 5% to about 10%, by weight of the depot dry weight.

The bisphosphonates of the invention are released from the depot formulations of the invention and from the compositions of the invention over a period of several weeks, e.g., about 4 weeks to 18 months.

Preferably, the bisphosphonate of the invention used to prepare the depot formulations is a very fine powder produced by any type of mircronization technique (e.g., jet milling or high pressure homogenization) having a particle of a size of about 0.1 microns to about 15 microns, preferably less than about 5 microns, even more preferably less than about 3 microns. It was found the micronizing the drug substance improved the encapsulation efficiency.

Further, the particle size distribution of the bisphosphonates of the invention may influence the release profile of the drug. Typically, the smaller the particle size, the lower is the burst and release during the first diffusion phase, e.g., the first 20 days. Preferably, particle size distribution is, e.g., x10<1.5 microns, i.e., 10% of the particles are smaller than 1 microns; x50<3 microns, i.e., 50% of the particles are smaller than 3 microns; or x 90<6 microns, i.e., 90% of the particles are smaller than 6 microns. Table I illustrates the salt forms of zoledronic acid when milled have an increased encapsulation efficiency and improved release profile when compared to unmilled salts forms of zoledronic acid.

TABLE I

| | Unmilled Drug | Milled Drug |
|---|---|---|
| | Substance | Substance |
| Batch no. | 6194.01 | 6470.01 |
| Salt type | Ca (1:2) | Ca (1:2) |
| Preparation | 10% drug load | 10% drug load |
| | 20% PLG/Glucose | 20% PLG/Glucose |
| | 0.5% PVA 18-88 | 0.5% PVA 18-88 |
| | Acetate buffer pH 4.0 | Acetate buffer pH 4.0 |
| Encapsulation efficiency | 27% | 68% |
| Initial release (24 h) | 41% | 13% |

In one embodiment, FIGS. 1 and 2 illustrates SEM images of unmilled and milled zoledronic acid as zinc salt. The samples were sputtered with gold-palladium and investigated by a scanning electron microscopy JEOL JSM6460 LV.

### II. Microparticles

It has been found that administration of microparticles comprising a low soluble salt of a bisphosphonate embedded in a biocompatible pharmacologically acceptable polymer, preferably a biodegradable pharmacologically acceptable polymer, suspended in a suitable vehicle gives release of all or of substantially all of the active agent over an extended period of time, e.g., one week up to 18 months, preferably for about 3 months to about 12 months.

The present invention in another aspect provides a process for the preparation of microparticles of the invention comprising:
(i) preparation of an internal organic phase comprising:
   (ia) dissolving the polymer or polymers in a suitable organic solvent or solvent mixture, and optionally dissolving/dispersing a porosity-influencing agent in the solution obtained in step (ia), or
      adding a basic salt to the solution obtained in step (ia),
      adding a surfactant to the solution obtained by step (ia);
   (ib) suspending the compound of the invention in the polymer solution obtained in step (ia), or dissolving the compound of the invention in a solvent miscible with the solvent used in step (ia) and mixing said solution with the polymer solution, or directly dissolving the compound of the invention in the polymer solution;
(ii) preparation of an external aqueous phase comprising
   (iia) preparing a buffer to adjust the pH to 3.0-5.0, e.g., acetate buffer, and
   (iib) dissolving a stabilizer in the solution obtained in step (iia);
(iii) mixing the internal organic phase with the external aqueous phase, e.g., with a device creating high shear forces, e.g., with a turbine or static mixer, to form an emulsion; and
(iv) hardening the microparticles by solvent evaporation or solvent extraction, washing the microparticles, e.g., with water, collecting and drying the microparticles, e.g., freeze-drying or drying under vacuum.

Suitable organic solvents for the polymers include, e.g., ethyl acetate, acetone, THF, acetonitrile, or halogenated hydrocarbons, e.g., methylene chloride, chloroform or hexafluoroisopropanol.

Suitable examples of a stabilizer for step (iib) include:
a) Polyvinyl alcohol (PVA), preferably having a weight average molecular weight from about 10,000 Da to about 150,000 Da, e.g., about 30,000 Da. Conveniently, the polyvinyl alcohol has low viscosity having a dynamic viscosity of from about 3 mPa s to about 9 mPa s when measured as a 4% aqueous solution at 20° C. or by DIN 53015.
   Suitably, the polyvinyl alcohol may be obtained from hydrolyzing polyvinyl acetate. Preferably, the content of the polyvinyl acetate is from about 10% to about 90% of the polyvinyl alcohol. Conveniently, the degree of hydrolysis is about 85% to about 89%. Typically the residual acetyl content is about 10-12%. Preferred brands include Mowiol® 4-88, 8-88 and 18-88 available from Kuraray Specialities Europe, GmbH.
   Preferably the polyvinyl alcohol is present in an amount of from about 0.1% to about 5%, e.g., about 0.5%, by weight of the volume of the external aqueous phase;
b) Hydroxyethyl cellulose (HEC) and/or hydroxypropyl cellulose (HPC), e.g., formed by reaction of cellulose with ethylene oxide and propylene oxide, respectively. HEC and HPC are available in a wide range of viscosity types; preferably the viscosity is medium. Preferred brands include Natrosol® from Hercules Inc., e.g., Natrosol® 250MR and Klucel® from Hercules Inc.
   Preferably, HEC and/or HPC is present in a total amount of from about 0.01% to about 5%, e.g., about 0.5%, by weight of the volume of the external aqueous phase;
c) Polyvinylpyrolidone, e.g., suitably with a molecular weight of between about 2,000 Da and 20,000 Da. Suitable examples include those commonly known as Povidone K12 F with an average molecular weight of about 2,500 Da, Povidone K15 with an average molecular weight of about 8,000 Da, or Povidone K17 with an average molecular weight of about 10,000 Da. Preferably, the polyvinylpyrolidone is present in an amount of from about 0.1% to about 50%, e.g., 10% by weight of the volume of the external aqueous phase
d) Gelatin, preferably porcine or fish gelatin. Conveniently, the gelatin has a viscosity of about 25 cps to about 35 cps for a 10% solution at 20° C. Typically pH of a 10% solution is from about 6 to about 7. A suitable brand has a high molecular weight, e.g., Norland high molecular weight fish gelatin obtainable from Norland Products Inc, Cranbury, N.J., USA.
   Preferably, the gelatin is present in an amount of from about 0.01% to about 5%, e.g., about 0.5%, by weight of the volume of the external aqueous phase.
   Preferably, polyvinyl alcohol is used. Preferably, no gelatin is used. Preferably, the microparticles are gelatin-free.

The resulting microparticles may have a diameter from a few submicrons to a few millimeters; e.g., diameters of at most, e.g., 10-200 microns, preferably 10-130 microns, more preferably 10-100 microns are strived for, e.g., in order to facilitate passage through an injection needle. A narrow particle size distribution is preferred. For example, the particle size distribution may be, e.g., x 10<20 microns, x 50<50 microns or x 90<80 microns.

Content uniformity of the microparticles and of a unit dose is excellent. Unit doses may be produced which vary from about 75% to about 125%, e.g., about 85% to about 115%, e.g., from about 90% to about 110%, or from about 95% to about 105%, of the theoretical dose.

The microparticles in dry state may, e.g., be mixed, e.g., coated, with an anti-agglomerating agent, or, e.g., covered by a layer of an anti-agglomerating agent, e.g., in a prefilled syringe or vial.

Suitable anti-agglomerating agents include, e.g., mannitol, glucose, dextrose, sucrose, sodium chloride or water soluble polymers, such as polyvinylpyrrolidone or polyethylene glycol, e.g., with the properties described above.

Preferably, an anti-agglomerating agent is present in an amount of about 0.1% to about 10%, e.g., about 4% by weight of the microparticles.

Prior to administration, the microparticies are suspended in a vehicle suitable for injection.

Accordingly, the present invention further provides a pharmaceutical composition comprising microparticles of the invention in a vehicle. The vehicle may optionally further contain:

a) one or more wetting agents; and/or

b) one or more tonicity agent; and/or

c) one or more viscosity increasing agents.

Preferably, the vehicle is water based, e.g., it may contain water, e.g., deionized, and optionally a buffer to adjust the pH to 7-7,5, e.g., a phosphate buffer, such as a mixture of Na₂HPO₄ and KH₂PO₄, and one or more of agents a), b) and/or c) as indicated above.

However, when using water as a vehicle, the microparticles of the invention may not suspend and may float on the top of the aqueous phase. In order to improve the capacity of the microparticles of the invention to be suspended in an aqueous medium, the vehicle preferably comprises a wetting agent a). The wetting agent is chosen to allow a quick and suitable suspendibility of the microparticles in the vehicle. Preferably, the microparticles are quickly wettened by the vehicle and quickly form a suspension therein.

Suitable wetting agents for suspending the microparticles of the invention in a water-based vehicle include non-ionic surfactants, such as poloxamers, or polyoxyethylene-sorbitan-fatty acid esters, the characteristics of which have been described above. A mixture of wetting agents may be used. Preferably, the wetting agent comprises Pluronic F68, Tween 20 and/or Tween 80.

The wetting agent or agents may be present in about 0.01% to about 1% by weight of the composition to be administered, preferably from 0.01-0.5% and may be present in about 0.01-5 mg/mL of the vehicle, e.g., about 2 mg/mL.

Preferably, the vehicle further comprises a tonicity agent b), such as mannitol, sodium chloride, glucose, dextrose, sucrose or glycerin. Preferably, the tonicity agent is mannitol.

The amount of tonicity agent is chosen to adjust the isotonicity of the composition to be administered. In case a tonicity agent is contained in the microparticles, e.g., to reduce agglomeration as mentioned above, the amount of tonicity agent is to be understood as the sum of both. For example, mannitol preferably may be from about 1% to about 5% by weight of the composition to be administered, preferably about 4.5%.

Preferably, the vehicle further comprises a viscosity increasing agent c). Suitable viscosity increasing agents include carboxymethyl cellulose sodium (CMC-Na), sorbitol, polyvinylpyrrolidone, or aluminum monostearate.

CMC-Na with a low viscosity may conveniently be used. Embodiments may be as described above. Typically, a CMC-Na with a low molecular weight is used. The viscosity may be of from about 1 mPa s to about 30 mPa s, e.g., from about 10 mPa s to about 15 mPa s when measured as a 1% (w/v) aqueous solution at 25° C. in a Brookfield LVT viscometer with a spindle 1 at 60 rpm, or a viscosity of 1-15 mPa*s for a solution of NaCMC 7LF (low molecular weight) as a 0.1-1% solution in water.

A polyvinylpyrrolidone having properties as described above may be used.

A viscosity increasing agent, e.g., CMC-Na, may be present in an amount of from about 0.1% to about 2%, e.g., about 0.7% or about 1.75% of the vehicle (by volume), e.g., in a concentration of about 1 mg/mL to about 30 mg/mL in the vehicle, e.g., about 7 mg/mL or about 17.5 mg/mL.

In a further aspect, the present invention provides a kit comprising microparticles of the invention and a vehicle of the invention. For example, the kit may comprise microparticles comprising the exact amount of compound of the invention to be administered, e.g., as described below, and about 1 mL to about 5 mL, e.g., about 2 mL of the vehicle of the invention.

In one embodiment, the dry microparticles, optionally in admixture with an anti-agglomerating agent, may be filled into a container, e.g., a vial or a syringe, and sterilized e.g., using gamma-irradiation. Prior to administration, the microparticles may be suspended in the container by adding a suitable vehicle, e.g., the vehicle described above. For example, the microparticles, optionally in admixture with an anti-agglomerating agent, a viscosity increasing agent and/or a tonicity agent, and the vehicle for suspension may be housed separately in a double chamber syringe. A mixture of the microparticles with an anti-agglomerating agent and/or a viscosity increasing agent and/or a tonicity agent, also forms part of the invention.

In another embodiment, under sterile conditions dry sterilized microparticles, optionally in admixture with an anti-agglomerating agent, may be suspended in a suitable vehicle, e.g., the vehicle described above, and filled into a container, e.g., a vial or a syringe. The solvent of the vehicle, e.g., the water, may then be removed, e.g., by freeze-drying or evaporation under vacuum, leading to a mixture of the microparticles and the solid components of the vehicle in the container. Prior to administration, the microparticles and solid components of the vehicle may be suspended in the container by adding a suitable vehicle, e.g., water, e.g., water for infusion, or preferably a low molarity phosphate buffer solution. For example, the mixture of the microparticles, optionally the anti-agglomerating agent, and solid components of the vehicle and the vehicle for suspension, e.g., water, may be housed separately in a double chamber syringe.

### III. Implants

It has been found that administration of implants comprising a low soluble salt of a bisphosphonate embedded in a biocompatible pharmacologically acceptable polymer gives release of all or of substantially all of the active agent over an extended period of time, e.g., one week up to 18 months, preferably for about 3 months to about 12 months.

The present invention in another aspect provides a process for the preparation of the implants of the invention comprising:
(i) preparation of a powder mixture of the poorly water soluble DS and the biodegradable polymer by cryo-milling with liquid nitrogen of both components together and/or using a organic solvent for a granulation step and removing this solvent again by a drying process;
(ii) filling a RAM extruder with the powder mixture (alternatively, a screw or a double-screw extruder is used);
(iii) heating the extruder walls to temperatures in the range of 50-120° C., in case of using poly(lactide-co-glycolide) as polymer matrix preferably 60-90° C.;
(iv) pushing the molten powder mixture through a pin hole of 1-4 mm diameter at small speed, preferably through a 1.5 mm pin hole with a speed of 5 mm/min.; and
(v) cutting the resulting sticks into shorter length depending on the anticipated dose, e.g., 20 mm.

For application the implants are placed in an applicator or trochar, sealed in aluminum foil and sterilized by using gamma-irradiation with a minimum dose of 25 kGy. These applicators are commercially available, e.g., by Rexam Pharma, Süddeutsche Feinmechanik GmbH (SFM) or Becton Dickerson.

### IV. Biocompatable Polymers

The polymer matrix of the depot formulations may be a synthetic or natural polymer. The polymer may be either a biodegradable or non-biodegradable or a combination of biodegradable and non-biodegradable polymers, preferably biodegradable.

By “polymer” is meant an homopolymer or a copolymer.

### Suitable Polymers Include:

(a) linear or branched polyesters which are linear chains radiating from a polyol moiety, e.g., glucose, e.g., a polyester, such as D-, L- or racemic polylactic acid, polyglycolic acid, polyhydroxybutyric acid, polycaprolactone, polyalkylene oxalate, polyalkylene glycol esters of an acid of the Kreb's cycle, e.g., citric acid cycle, and the like or a combination thereof,
(b) polymers or copolymers of organic ethers, anhydrides, amides and orthoesters, including such copolymers with other monomers, e.g., a polyanhydride, such as a copolymer of 1,3-bis-(p-carboxyphenoxy)-propane and a diacid, e.g., sebacic acid, or a copolymer of erucic acid dimer with sebacic acid; a polyorthoester resulting from reaction of an ortho-ester with a triol, e.g., 1,2,6-hexanetriol, or of a diketene acetal, e.g., 3,9-diethylidene-2,4,8,10-tetraoxaspiro[5,5]un-decane, with a diol, e.g., 1,6-dihexanediol, triethyleneglycol or 1,10-decanediol; or a polyester amide obtained with an amide-diol monomer, e.g., 1,2-di-(hydroxyacetamido)-ethane or 1,10-di-(hydroxyacetamido)decane; or
(c) polyvinylalcohol.

The polymers may be cross-linked or non-cross-linked, usually not more than 5%, typically less than 1%.

Table II lists examples of the polymers of the invention:

TABLE II

| Product Name | Polymer |
|---|---|
| D,L-POLYMI/D-GLUCOSE | Star-branched |
| | Poly(D,L-lactide-co-glycolide) |
| | 50:50/D-Glucose |
| Resomer ® R 202 H | Linear |
| | Poly(D,L-lactide) |
| | free carboxylic acid end group |
| Resomer ® R 202 | Linear |
| | Poly(D,L-lactide) |
| Resomer ® R 203 | Linear |
| | Poly(D,L-lactide) |
| Resomer ® RG 752 | Linear |
| | Poly(D,L-lactide-co-glycolide) |
| | 75:25 |
| Resomer ® RG 753 S | Linear |
| | Poly(D,L-lactide-co-glycolide) |
| | 75:25 |
| Lactel ® 100D020A | Linear |
| | Poly(D,L-lactide) |
| | free carboxylic acid end group |
| Lactel ® 100D040A | Linear |
| | Poly(D,L-lactide) |
| | free carboxylic acid end group |
| Lactel ® 100D040 | Linear |
| | Poly(D,L-lactide) |
| Lactel ® 100D065 | Linear |
| | Poly(D,L-lactide) |
| Lactel ® 85DG065 | Linear |
| | Poly(D,L-lactide-co-glycolide) |
| | 85:15 |
| Lactel ® 75DG065 | Linear |
| | Poly(D,L-lactide-co-glycolide) |
| | 75:25 |
| Lactel ® 65DG065 | Linear |
| | Poly(D,L-lactide-co-glycolide) |
| | 65:35 |
| Lactel ® 50DG065 | Linear |
| | Poly(D,L-lactide-co-glycolide) |
| | 50:50 |
| Lactel ® 50DG085 | Linear |
| | Poly(D,L-lactide-co-glycolide) |
| | 50:50 |
| Lactel ® 50DG105 | Linear |
| | Poly(D,L-lactide-co-glycolide) |
| | 50:50 |
| Medisorb ® 100 DL HIGH IV | Linear |
| | Poly(D,L-lactide) |
| Medisorb ® 100 DL LOW IV | Linear |
| | Poly(D,L-lactide) |
| Medisorb ® 8515 DL HIGH IV | Linear |
| | Poly(D,L-lactide-co-glycolide) |
| | 85:15 |
| Medisorb ® 8515 DL LOW IV | Linear |
| | Poly(D,L-lactide-co-glycolide) |
| | 85:15 |
| Medisorb ® 7525 DL HIGH IV | Linear |
| | Poly(D,L-lactide-co-glycolide) |
| | 75:25 |
| Medisorb ® 7525 DL LOW IV | Linear |
| | Poly(D,L-lactide-co-glycolide) |
| | 75:25 |
| Medisorb ® 6535 DL HIGH IV | Linear |
| | Poly(D,L-lactide-co-glycolide) |
| | 65:35 |
| Medisorb ® 6535 DL LOW IV | Linear |
| | Poly(D,L-lactide-co-glycolide) |
| | 65:35 |
| Medisorb ® 5050 DL HIGH IV | Linear |
| | Poly(D,L-lactide-co-glycolide) |
| | 50:50 |
| Medisorb ® 5050 DL LOW IV | Linear |
| | Poly(D,L-lactide-co-glycolide) |
| | 50:50 |

The preferred polymers of this invention are linear polyesters and branched chain polyesters. The linear polyesters may be prepared from alpha-hydroxy carboxylic acids, e.g., lactic acid and/or glycolic acid, by condensation of the lactone dimers. The preferred polyester chains in the linear or branched (star) polymers are copolymers of the alpha-carboxylic acid moieties, lactic acid and glycolic acid, or of the lactone dimers. The molar ratio of lactide: glycolide of polylactide-co-glycolides in the linear or branched polyesters is preferably from about 100:0 to 40:60, more preferred from. 95:5 to 50:50, most preferred from 85:15 to 65:35.

Linear polyesters, e.g., linear polylactide-co-glycolides (PLG), preferably used according to the invention have a weight average molecular weight (Mw) between about 10,000 Da and about 500,000 Da,.e.g., about 50,000 Da. Such polymers have a polydispersity M_{w}/Mₙ, e.g., between 1.2 and 2. Suitable examples include, e.g., poly(D,L-lactide-co-glycolide), linear poly (D,L-lactide) and liner-poly (D,L-lactide) free carboxylic acid end group, e.g., having a general formula —[(C₆H₈O₄)ₓ(C₄H₄O₄)_{y}]ₙ— (each of x, y and n having a value so that the total sum gives the above indicated Mws), e.g., those commercially-available, e.g., Resomers® from Boehringer Ingelheim, Lactel® from Durect, Purasorb® from Purac and Medisorb® from Lakeshore.

Branched polyesters, e.g., branched polylactide-co-glycolides, also used according to the invention may be prepared using polyhydroxy compounds, e.g., polyol, e.g., glucose or mannitol as the initiator. These esters of a polyol are known and described, e.g., in GB 2,145,422 B, the contents of which are incorporated herein by reference. The polyol contains at least 3 hydroxy groups and has a molecular weight of up to 20,000 Da, with at least 1, preferably at least 2, e.g., as a mean 3 of the hydroxy groups of the polyol being in the form of ester groups, which contain poly-lactide or co-poly-lactide chains. Typically 0.2% glucose is used to initiate polymerization. The branched polyesters (Glu-PLG) have a central glucose moiety having rays of linear polylactide chains, e.g., they have a star shaped structure.

The branched polyesters having a central glucose moiety having rays of linear polylactide-co-glycolide chains (Glu-PLG) may be prepared by reacting a polyol with a lactide and preferably also a glycolide at an elevated temperature in the presence of a catalyst, which makes a ring opening polymerization feasible.

The branched polyesters having a central glucose moiety having rays of linear polylactide-co-glycolide chains (Glu-PLG) preferably have an weight average molecular weight M_{w} in the range of from about 10,000-200,000, preferably 25,000-100,000, especially 35,000-60,000, e.g., about 50,000 Da, and a polydispersity, e.g., of from 1.7-3.0, e.g., 2.0-2.5. The intrinsic viscosities of star polymers of M_{w} 35,000 or M_{w} 60,000 are 0.36 dL/g or 0.51 dL/g, respectively, in chloroform. A star polymer having a M_{w} 52,000 has a viscosity of 0.475 dl/g in chloroform.

The desired rate of degradation of polymers and the desired release profile for compounds of the invention may be varied depending on the kind of monomer, whether a homo- or a copolymer or whether a mixture of polymers is employed.

### V. Method of Treatment

The uses and methods of the present invention represent an improvement to existing therapy of malignant diseases in which bisphosphonates are used to prevent or inhibit development of bone metastases or excessive bone resorption, and also for the therapy of inflammatory diseases such as rheumatoid arthritis and osteoarthritis. Use of bisphosphonates to embolise newly-formed blood vessels has been found to lead to suppression of tumors, e.g., solid tumors, and metastastes, e.g., bone metastases and even reduction in size of tumors, e.g., solid tumors, and metastases, e.g., bone metastases, after appropriate periods of treatment. It has been observed using angiography that newly-formed blood vessels disappear after bisphosphonate treatment, but that normal blood vessels remain intact. Further it has been observed that the embolised blood vessels are not restored following cessation of the bisphosphonate treatment. Also it has been observed that bone metastasis, rheumatoid arthritis and osteoarthritis patients experience decreased pain following bisphosphonate treatment.

Conditions of abnormally increased bone turnover which may be treated in accordance with the present invention include: treatment of postmenopausal osteoporosis, e.g., to reduce the risk of osteoporotic fractures; prevention of postmenopausal osteoporosis, e.g., prevention of postmenopausal bone loss; treatment or prevention of male osteoporosis; treatment or prevention of corticosteroid-induced osteoporosis and other forms of bone loss secondary to or due to medication, e.g., diphenylhydantoin, thyroid hormone replacement therapy; treatment or prevention of bone loss associated with immobilisation and space flight; treatment or prevention of bone loss associated with rheumatoid arthritis, osteogenesis imperfecta, hyperthyroidism, anorexia nervosa, organ transplantation, joint prosthesis loosening, and other medical conditions. For example, such other medical conditions may include treatment or prevention of periarticular bone erosions in rheumatoid arthritis; treatment of osteoarthritis, e.g., prevention/treatment of subchondral osteosclerosis, subchondral bone cysts, osteophyte formation, and of osteoarthritic pain, e.g., by reduction in intra-osseous pressure; treatment or prevention of hypercalcemia resulting from excessive bone resorption secondary to hyperparathyroidism, thyrotoxicosis, sarcoidosis, or hypervitaminosis D, dental resorptive lesions, pain associated with any of the above conditions, particularly, osteopenia, Paget's disease, osteoporosis, rheumatoid arthritis, osteoarthritis.

Appropriate dosage of the depot formulations of the invention will of course vary, e.g., depending on the condition to be treated (e.g., the disease type or the nature of resistance), the drug used, the effect desired and the mode of administration.

In general, satisfactory results are obtained on administration, e.g., parenteral administration, at dosages on the order of from about 0.2 mg to about 100 mg, e.g., 0.2 mg to about 35 mg, preferably from about 3 mg to about 100 mg of the compound of the invention per injection per month or about 0.03 mg to about 1.2 mg, e.g., 0.03-0.3 mg per kg animal body weight per month. Suitable monthly dosages for patients are thus in the order of about 0.3 mg to about 100 mg of a compound of the invention, preferably a compound of formula (1).

The properties of the depot formulation and the compositions of the invention may be tested in standard animal tests or clinical trials.

The depot formulation and the compositions of the invention are well-tolerated.

The following Examples serve to illustrate the invention, without any limitation.

### Example 1

### Manufacturing Process for Making Microparticles with 5% of Zoledronic Acid in the Calcium Salt Form

6.26 g of PLGA 75:25 (IV 0.68 dL/g) are dissolved in 44.25 g dichloromethane. 0.43 g of micronized calcium zoledronate (1:2 salt) are suspended in this solution by using a rotor-stator high shear mixer at 20′000 rpm for 1 minute under cooling (ca. 10° C.). This suspension is then mixed with a 0.5% polyvinyl alcohol 18-88 solution containing 19 mM acetate buffer in a volumetric ratio of 1:20 through an in-line rotor-stator high shear mixer at 4500 rpm with flow rates of 10:200 mL/min. The resulting emulsion is collected in a double walled reactor which is then heated up from 20-54° C. in 3.5 hours under stirring with a propeller blade stirrer at 400 rpm. The emulsion is heated for further 30 minutes at 54° C. before it is cooled down to room temperature and stirring is stopped. Through this process solid microparticles are formed out of the emulsion troplets. The isolation of the microparticles is done by sedimentation and decantation and filtration. The microparticles are washed with water on the filter and are finally dried in vacuum. The dried microparticles are sieved through 140 micron and sterilized by gamma-irradiation with 30 kGy. 5.55 g (82.9%) of microparticles were yielded. The particle size distribution is as follows: 10% smaller than 15.4 micron, 50% smaller than 39.0 micron, 90% smaller than 59.6 micron. The assay is found to be 4.5% which corresponds to an encapsulation rate of 90%. The in vitro drug release is shown in FIG. 3.

### Example 1A

### Manufacturing Process for Making Microparticles with 5% of Zoledronic Acid in the Calcium Salt Form

6.57 g of PLGA 75:25 (IV 0.68 dL/g) is dissolved in 43.6 g dichloromethane. 0.43 g of micronized calcium zoledronate (1:2 salt) is suspended in this solution by using a rotor-stator high shear mixer at 20′000 rpm for 1 minute under cooling (ca. 10° C.). This suspension is then mixed with a 0.5% polyvinyl alcohol 18-88 solution containing 100 mM acetate buffer in a volumetric ratio of 1:20 through an in-line rotor-stator high shear mixer at 4000 rpm with flow rates of 30:600 mL/min. The resulting emulsion is collected in a double walled reactor which is then heated up from 20-54° C. in 5 hours under stirring with a propeller blade stirrer at 400 rpm. The emulsion is heated for further 2 hours at 54° C. before it is cooled down to room temperature and stirring is stopped. Through this process solid microparticles are formed out of the emulsion troplets. The isolation of the microparticles is done by sedimentation and decantation and filtration. The microparticles are washed with water on the filter and are finally dried in vacuum. The dried microparticles are sieved through 140 micron and sterilized by gamma-irradiation with 30 kGy. 4.92 g (71%) of microparticles were yielded. The particle size distribution is as follows: 10% smaller than 21.8 micron, 50% smaller than 48.5 micron, 90% smaller than 73.2 micron. The assay is found to be 4.5% which corresponds to an encapsulation rate of 90%. The in vitro drug release is shown in FIG. 6 (This example is Batch no. 8370.03, light blue curve).

### Example 2

### Manufacturing Process for Making Microparticles with 10% of Zoledronic Acid in the Calcium Salt Form

In the same manner as described in Example 1, 7.70 g PLGA 75:25 (IV 0.68 dL/g), 51.07 g dichloromethane and 1.23 g micronized calcium zoledronate (1:2 salt) are used to prepare microparticles with a yield of 7.15 g (80.0%). The particle size distribution is found as follows: 10% smaller than 20.4 micron, 50% smaller than 45.3 micron, 90% smaller than 69.9 micron. The assay is found to be 9.3% which corresponds to an encapsulation rate of 93%. The in vitro drug release is shown in FIG. 3.

### Example 3

### Manufacturing Process for Making Microparticles with 15% of Zoledronic Acid in the Calcium Salt Form

In the same manner as described in Example 13.69 g PLGA 75:25 (IV 0.68 dL/g), 28.28 g dichloromethane and 0.92 g micronized calcium zoledronate (1:2 salt) are used to prepare microparticles with a yield of 3.17 g (69%). The particle size distribution is found as follows: 10% smaller than 16.0 micron, 50% smaller than 39.0 micron, 90% smaller than 64.1 micron. The assay is found to be 11.6% which corresponds to an encapsulation rate of 77.3%. The in vitro drug release is shown in FIG. 3.

### Example 4

### Manufacturing Process for Making Microparticles with 15% of Zoledronic Acid in the Zinc Salt Form

In the same manner as described in Example 1, 3.70 g PLGA 75:25 (IV 0.68 dL/g), 27.97 g dichloromethane and 0.88 g micronized zinc zoledronate (1:2 salt) are used to prepare microparticles with a yield of 3.11 g (68%). The particle size distribution is found as follows: 10% smaller than 19.4 micron, 50% smaller than 45.1 micron, 90% smaller than 79.0 micron. The assay is found to be 13.9% which corresponds to an encapsulation rate of 92.7%. The in vitro drug release is shown in FIG. 4.

### Example 5

### Manufacturing Process for Making Microparticles with 20% of Zoledronic Acid in the Zinc Salt Form

In the same manner as described in Example 15.03 g PLGA 75:25 (IV 0.68 dL/g), 35.51 g dichloromethane and 1.64 g micronized zinc zoledronate (1:2 salt) are used to prepare microparticles with a yield of 5.85 g (88%). The particle size distribution is found as follows: 10% smaller than 17.2 micron, 50% smaller than 43.6 micron, 90% smaller than 66.0 micron. The assay is found to be 17.3% which corresponds to an encapsulation rate of 86.5%. The in vitro drug release is shown in FIG. 4.

### Example 6

### Manufacturing Process for Making Microparticles with Unmilled and Milled Drug Substance

Before milling the drug substance particle size distribution is as the following: 10% smaller than 10.6 micron, 50% smaller than 177.77 micron and 90% smaller than 745.7 micron. After jet-milling with 6 bar the particle size distribution is as the following: 10% smaller than 1.0 micron, 50% smaller than 2.5 micron and 90% smaller than 5.4 micron.

Using the process as described in Example 2 with the difference that for this example a star-branched PLGA 55:45 is used, the encapsulation rate with the not-milled drug substance is only 27% and the initial release (within 24 hours) is very high (41%). Using the micronized drug substance the encapsulation rate is significantly higher (68%) and the initial release was rather low with only 13%.

### Example 7

### Vehicle for Microspheres

7 g sodium carboxy methlycellulose (CMC-Na), 45 g D-mannitol and 2 g Pluronic F68 is dissolved in about 0.9 L hot deionized water of a temperature of about 90° C. under strong stirring with a magnetic stirrer. The resulting clear solution is cooled to 20° C. and filled up with deionized water to 1.0 L.

### Example 8

### Reconstitution of the Microparticles

500 mg of microparticles of Examples 1-6 are suspended in 2.0 mL of the vehicle of Example 7 in 6R vials. The suspensions are homogenized by shaking for about 30 seconds. The reconstituted suspension may be injected without any issues using a 20 gauge needle.

### Example 9

### Tolerability Study of Calcium Zoledronate Microparticles in Rats after s.c. Administration

The microparticles of Examples 2 and 3 are suspended in a vehicle containing sodium carboxy methylcellulose, D-mannitol, Pluronics F68 and water for injection. 200 microliters of these suspensions were injected subcutaneously to the shaved skin at the left dorsal side of 8 weeks old female virgin Wistar rats (body weight approximately 220 g). In this way 1 mg dose (per animal) of zoledronic acid of the 15% calcium zoledronate microparticles (Example 3) and 2 mg dose (per animal) of zoledronic acid of the 10% calcium zoledronate microparticles (Example 2) were applied to a group of 6 animals per formulation. The thickness of the skin will be measured by a micro-caliper at the side of injection and the contra-lateral non-injected side. As reference a suspension of non-encapsulated drug substance is injected in a dose of 60 microgram. In addition, placebo microparticles made out of PLGA 75:25 (IV 0.68 dL/g) are also injected as control.

The results are shown in FIG. 5. The non-encapsulated drug substance caused a strong swelling of the skin demonstrating the high local skin irritation caused by the zoledronic acid. In contrast, the encapsulated drug substance caused only a slight increase in skin thickness. However, the slightly increased thickness is observed for over 80 days indicating a constant release of zoledronic acid out of the depot. The placebo microparticles did not show any significant skin irritation.

### Example 10

### The Production of Implants with 10% Zoledronic Acid as Calcium Salt

1.5 g of the micronized calcium zoledronate (1:2 salt) and 10.7 g of a PLGA 65:35 (IV 0.65 dL/g) are mixed thoroughly through cryo-milling in liquid nitrogen. The resulting fine powder are extruded at 90° C. through a ram extruder with a speed of 5 mm/min. The implants of 1.5 mm diameter are cut at 2 cm length. An extrusion yield of 76% are achieved resulting in a number of 211 implants. The implants are placed in applicators, sealed in aluminum foil and finally sterilized by using gamma-irradiation with a dose of 30 kGy. The in vitro drug release test reveals a continuous release of the zoledronic acid for more than 70 days.

1. Microparticles comprising a low soluble salt of a bisphosphonate of compound of formula (I): 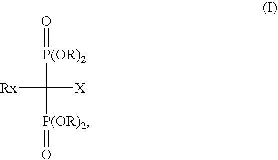
wherein
X is hydrogen, hydroxyl, amino, alkanoyl, or an amino group substituted by C₁-C₄ alkyl, or alkanoyl;
R is hydrogen or C₁-C₄ alkyl; and
Rx is a side chain which contains an optionally substituted amino group, or a nitrogen containing heterocycle (including aromatic nitrogen-containing heterocycles),
or a pharmaceutically acceptable salt thereof or any hydrate thereof, embedded in a polymer matrix, wherein the pharmaceutically acceptable salt thereof is selected from calcium, magnesium, and zinc; and a polymer matrix.

2. Microparticles according to claim 1, wherein the bisphosphonate is 2-(imidazol-1yl)-1-hydroxyethane-1,1-diphosphonic acid.

3. Microparticles according to claim 1, wherein pharmaceutically acceptable salt is calcium.

4. Microparticles according to claim 1, wherein pharmaceutically acceptable salt is zinc.

5. Microparticles according to claim 1, wherein the polymer matrix comprises a linear or branched polylactide-co-glycolide.

6. Microparticles according to claim 1, further comprising a surfactant, a porosity influencing agent and/or a basic salt.

7. A pharmaceutical composition comprising the microparticles according to claim 1 and a water-based vehicle comprising a wetting agent.

8. A pharmaceutical composition according to claim 6, wherein the wetting agent comprises a poloxamer and/or a polyoxyethylene-sorbitan-fatty acid ester.

9. A composition according to claim 6, wherein the vehicle comprises a tonicity agent.

10. A composition according to claim 6, wherein the vehicle comprises a viscosity increasing agent.

11. A kit comprising microparticles according to claim 1 and a water-based vehicle.

12. (canceled)

13. A method of treating a disease or disorder in a subject in need thereof which comprises administering microparticles according to claim 1 to the subject.

14. An implant device comprising a low soluble salt of a bisphosphonate of compound of formula (I): 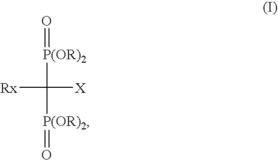
wherein
X is hydrogen, hydroxyl, amino, alkanoyl, or an amino group substituted by C₁-C₄ alkyl, or alkanoyl;
R is hydrogen or C₁-C₄ alkyl; and
Rx is a side chain which contains an optionally substituted amino group, or a nitrogen containing heterocycle (including aromatic nitrogen-containing heterocycles),
or a pharmaceutically acceptable salt thereof or any hydrate thereof, embedded in a polymer matrix, wherein the pharmaceutically acceptable salt thereof; embedded in a polymer matrix, wherein the pharmaceutically acceptable salt is selected from calcium, magnesium, and zinc; and a polymer matrix.

15. An implant device according to claim 1, wherein the bisphosphonate is 2-(imidazol-1yl)-1-hydroxyethane-1,1-diphosphonic acid.

16. An implant device according to claim 1, wherein the pharmaceutically acceptable salt thereof is calcium.

17. An implant device according to claim 1, wherein the pharmaceutically acceptable salt thereof is zinc.

18. An implant device according to claim 1, wherein the polymer matrix comprises a linear or branched polylactide-co-glycolide.

19. An implant device according to claim 1, further comprising a surfactant, a porosity influencing agent and/or a basic salt.

20. A process for the preparation of microparticles according to claim 1, comprising the steps of:
(a) preparing an internal organic phase comprising by dissolving the polymer or polymers in a suitable organic solvent or solvent mixture, and optionally dissolving/dispersing a porosity-influencing agent in the solution, or adding a basic salt to the solution to form a polymer solution;
(b) adding a surfactant and suspending the bisphosphonate in the polymer solution, or dissolving the compound of the invention in a solvent miscible with the solvent used in step (a) and mixing said solution with the polymer solution, or directly dissolving the compound of the invention in the polymer solution;
(c) of an external aqueous phase comprising preparing a buffer to adjust the pH to 3.0-5.0, e.g., acetate buffer and dissolving a stabilizer;
(d) mixing the internal organic phase with the external aqueous phase to form an emulsion;
(e) hardening the microparticles by solvent evaporation or solvent extraction; and
(f) washing, collecting and drying the microparticles.

21. A process for the preparation of implants according to claim 14, comprising the steps of:
(i) preparing a powder mixture of a poorly water soluble DS and a biodegradable polymer by cryo-milling with liquid nitrogen;
(ii) filling an extruder with the powder mixture;
(iii) heating the extruder walls to temperatures in the range of 50-120° C.;
(iv) pushing the molten powder mixture through a pin hole of 1-4 mm diameter at a speed of 5 mm/min; and
(v) cutting the resulting sticks into shorter length depending on the anticipated dose.
